(19) United States
(12) Patent Application Publication
(10) Pub. No.: US 2024/0293192 A1
(43) Pub. Date: Sep. 5, 2024

(54) VISUALIZATION ROBOT WITH OPHTHALMIC SURGERY-OPTIMIZED KINEMATICS

(71) Applicant: Alcon Inc., Fribourg (CH)

(72) Inventor: Steven T. Charles, Memphis, TN (US)

(73) Assignee: Alcon Inc., Fribourg (CH)

(21) Appl. No.: 18/592,640

(22) Filed: Mar. 1, 2024

Related U.S. Application Data
(60) Provisional application No. 63/488,083, filed on Mar. 2, 2023.
(60) Provisional application No. 63/504,273, filed on May 25, 2023.

(51) Int. Cl. A61B 34/30; A61B 34/20; G02B 21/00

(52) U.S. Cl. CPC A61B 34/30; A61B 34/20; G02B 21/0012; A61B 2034/2055

(57) ABSTRACT

A robotic system includes a base, a support column, and a selective-compliance-articulated robot arm (SCARA) connected to the base via the support column. The SCARA is configured with ophthalmic visualization-specific kinematics. The SCARA includes first, second, and third revolute joints. A first link is connected to the base via the first revolute joint. A second link is connected to the first link via the second revolute joint, has a distal end connected to the third revolute joint, and is configured as a four-bar mechanism. The second link is connectable to a microscope. A linear actuator is connected to the second link. In response to electronic control signals from an electronic control unit, the linear actuator controls vertical motion of the SCARA. None of the revolute joints of the SCARA functions in an anti-gravity mode.

### CROSS-REFERENCE TO RELATED APPLICATIONS

The present application claims the benefit of priority to U.S. Provisional Application No. 63/488,083 filed Mar. 2, 2023, and U.S. Provisional Application No. 63/504,273 filed May 25, 2023, which are hereby incorporated by reference in their respective entireties.

### INTRODUCTION

Serial robots are used in a wide range of industrial and medical applications. A typical serial robot includes an articulated arm having multiple rigid bars, segments, or links. The links are interconnected via revolute joints to form an open kinematic chain. As appreciated in the art, a robot arm having open-chain kinematics includes an arrangement of successive links and joints in which a distalmost one of links is freely moveable within a well-defined operating space. High stiffness is provided through the constituent joints, with each respective joint being either actuated (“actively driven”) or unactuated (“passively moveable”) to control joint angles and the relative motion/orientation of the interconnected links.

In a serial robot arm of a type used to support a digital or analog microscope for visualization during an ophthalmic procedure, the distalmost link of the serial robot arm may be securely connected to such a microscope via a suitable end-effector. For example, the serial robot arm could be connected to an optical head of the microscope via a mounting bracket. Motion of the serial robot arm through its available motion degrees of freedom ultimately enables the end-effector and the connected microscope to reach a desired position and orientation in free space, such as when positioning the above-noted optical head relative to a patient's head or body within an ophthalmic surgical suite.

### SUMMARY

Disclosed herein is robotic system having a serial robot arm. The robot arm, which has five degrees of freedom in the illustrated embodiments, is constructed in accordance with predetermined surgical task-specific kinematics. In particular, the kinematics are optimized for supporting and positioning of a digital or analog microscope for visualization of ocular anatomy attendant to ophthalmic procedures, e.g., cataract or vitreoretinal surgical applications. A typical serial robot arm used in such an environment can experience problematic singularities, potential instability, and payload limitations. The optimized kinematics contemplated herein are therefore specially geared toward solving the types of payload positioning problems frequently encountered by eye surgeons when positioning a microscope with the assistance of a serial robot arm.

In accordance with an aspect of the disclosure, the robotic system described herein includes a selective-compliance articulated robot arm (SCARA) connected to a base. The SCARA in turn includes multiple links and joints. Among the constituent links, a first link is connected to the base via a first one of the joints (“first revolute joint”). A second link, which is connected to the first link via a second one of the joints (“second revolute joint”), has a distal end that is connected to a third one of the joints (“third revolute joint”). The second link in this particular embodiment is configured as a four-bar mechanism, with the four-bar mechanism being connectable to an ophthalmic microscope. A linear actuator, e.g., a non-back drivable vertical harmonic drive unit, may be connected to the second link to control vertical motion of the second link, and thus of the connected microscope. As a defining characteristic of the SCARA, none of the revolute joints of the SCARA functions in an anti-gravity mode.

Embodiments of the robotic system may include a plurality of harmonic drive units, with at least one of the revolute joints of the SCARA being powered by a corresponding one of the harmonic drive units in response to the electronic control signals. For example, each of the revolute joints of the SCARA could be individually powered by a corresponding one of the harmonic drive units in response to the electronic control signals.

The robotic system may include the microscope and a bracket mounted to the distal end of the second link. The microscope in such an embodiment could be mounted to the bracket. The microscope may be embodied as a digital or analog ophthalmic microscope having an optical head. In such a configuration, the base may be positioned on a floor, with the optical head having a pitch axis arranged parallel to the floor. The pitch and rotation of the optical head could be selectively lockable. A position sensor may be connected to the optical head and in communication with the ECU. An optional gas spring counterbalance device is operatively connected to the second link in some implementations.

An aspect of the disclosure includes the first link and the second link being constructed of a suitable material, e.g., a powdered metallurgy alloy-like material made using cold isostatic pressing and possibly containing aluminum and beryllium.

Also disclosed herein is a SCARA for use with a microscope and a base. An embodiment of the SCARA includes a plurality of revolute joints, including a first revolute joint, a second revolute joint, and a third revolute joint, along with a first link connectable to the base via the first revolute joint. A second link is connected to the first link via the second revolute joint, has a distal end connected to the third revolute joint, and is configured as a four-bar mechanism. The second link is configured to connect to the microscope. A linear actuator is connected in this embodiment to the second link and configured to control vertical motion thereof in response to electronic control signals from an ECU. The linear actuator includes a non-back drivable vertical harmonic drive unit, and wherein none of the revolute joints of the SCARA functions in an anti-gravity mode.

The above-described features and advantages and other possible features and advantages of the present disclosure will be apparent from the following detailed description of the best modes for carrying out the disclosure when taken in connection with the accompanying drawings.

### BRIEF DESCRIPTION OF THE DRAWINGS

FIG. 1 is an illustration of an exemplary ophthalmic surgical suite having a robotic system configured as set forth herein.

FIG. 2 is a top view illustration of a selective-compliance articulated robot arm (SCARA) that is usable as part of the exemplary robotic system shown in FIG. 1.

FIG. 3 is a side view illustration of the representative embodiment of the SCARA depicted in FIG. 2.

FIG. 4 illustrates an exemplary payload in the form of an ophthalmic microscope that is connectable to a distal end of the SCARA shown in FIGS. 2 and 3.

The solutions of the present disclosure may be modified or presented in alternative forms. Representative embodiments are shown by way of example in the drawings and described in detail below. However, inventive aspects of this disclosure are not limited to the disclosed embodiments. Rather, the present disclosure is intended to cover alternatives falling within the scope of the disclosure as defined by the appended claims.

### DETAILED DESCRIPTION

Embodiments of the present disclosure are described herein. It is to be understood, however, that the disclosed embodiments are merely examples and other embodiments can take various and alternative forms. The figures are not necessarily drawn to scale. Some features could be exaggerated or minimized to show details of particular components. Therefore, specific structural and functional details disclosed herein are not to be interpreted as limiting, but merely as a representative basis for teaching one skilled in the art to variously employ the present disclosure.

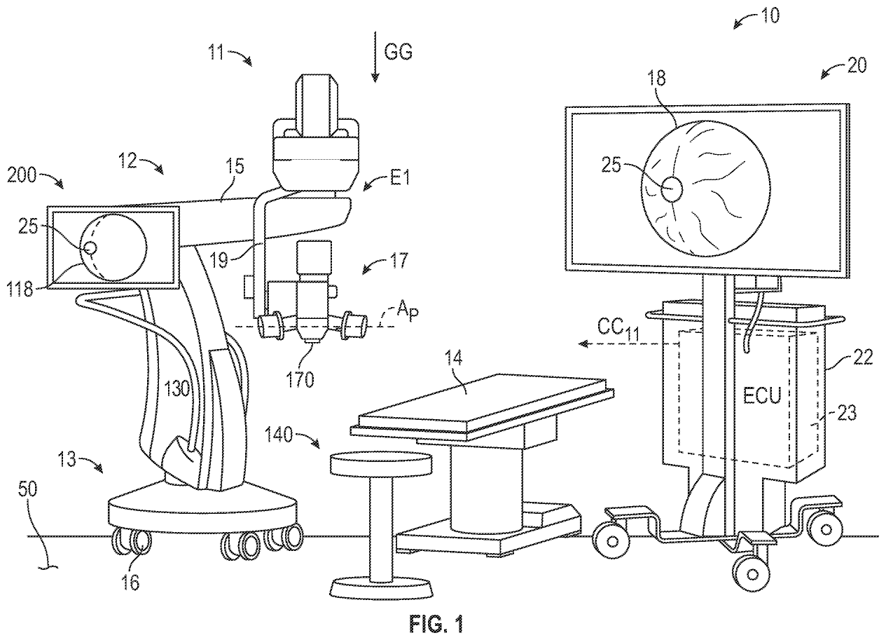

Referring to the drawings, wherein like reference numbers refer to like components, a representative ophthalmic surgical suite 10 is illustrated in FIG. 1. The ophthalmic surgical suite 10 is equipped with a robotic system 11 having a multi-axis visualization robot 12 and an operating table 14. During performance of a vitreoretinal, cataract, or other ocular surgical procedure within the suite 10, a patient (not shown) may be positioned on the operating table 14 or on another suitable platform, with a surgeon (not shown) seated on a stool 140. Although omitted from FIG. 1 for the purpose of illustrative simplicity, respective heights of the operating table 14 and the stool 140 may be adjusted with the assistance of automatic and/or manual knobs, levers, or foot pedals in a typical implementation.

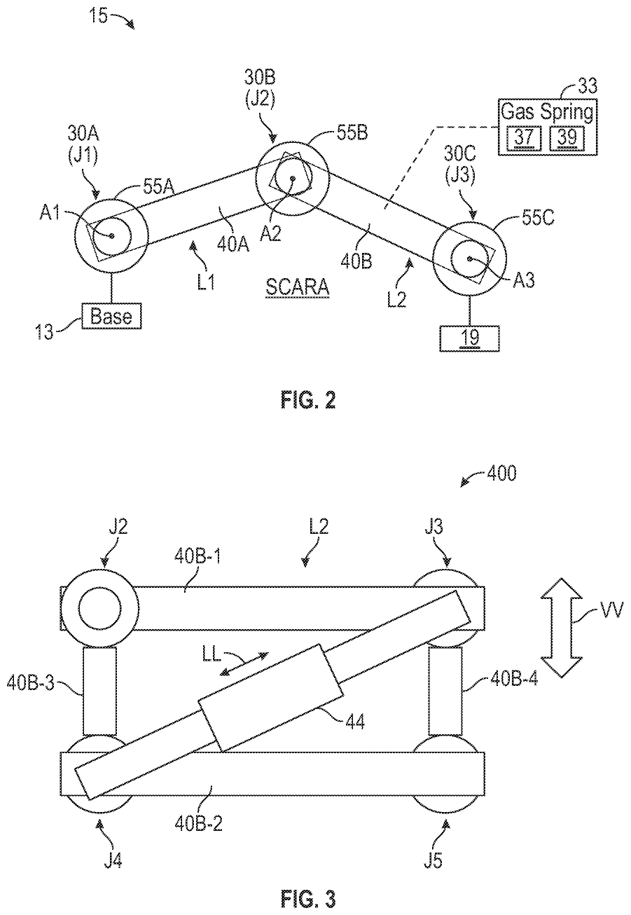

The visualization robot 12 includes a base 13 mounted or positioned relative to a floor 50 of the ophthalmic surgical suite 10, e.g., directly or via a mobile platform having lockable wheels 16 as shown. The base 13 in the illustrated exemplary embodiment of FIG. 1 is connected to a selective-compliance articulated robot arm (SCARA) 15 via an intervening support column 130, with the SCARA 15 constructed with visualization application-specific kinematics as set forth below with particular reference to FIGS. 2, 3, and 4. In general, the SCARA 15 is configured as an articulated serial robot arm having improved performance capabilities relative to typical serial robots when used to support visualization equipment preparatory to or in conjunction with eye surgeries when performed within the suite 10. At least some of the attendant improvements include the elimination of relevant singularities, improved stability, and more robust handling of relatively heavy or bulky payloads, e.g., an ophthalmic microscope 17 and its attached optical head 170. Another key attribute is that the SCARA 15 and a connected payload do not drop or fall down when power is lost or turned off.

A singularity that is commonly experienced in a surgical suite, such as the exemplary ophthalmic surgical suite 10 of FIG. 1, can prevent a high-resolution display screen 20, e.g., a high-resolution medical display screen, from being positioned directly in front of the attending surgeon. This in turn requires a turn of the surgeon's head in order to properly view the display screen 20 during visualization. Another common singularity results in interference with free motion of a robot arm. This is often caused by contact between such a robot arm and the patient's chest when the surgeon moves the robot arm to thereby position the optical head 170. The SCARA 15 as described in detail herein is therefore intended to address these and other positioning problems commonly associated with serial robot arms, thus improving upon the current state of the art of robot-assisted visualization.

In particular, the application-specific kinematics of the SCARA 15 of FIGS. 1-4 as contemplated herein are specific to visualization efforts performed in support of cataract, glaucoma, cornea and vitreoretinal (“VR”) surgeries. A distal end E1 of a typical serial robot (not shown) is securely connected to the optical head 170 through which the surgeon views a patient's ocular anatomy under high magnification. For instance, using associated hardware and software, the surgeon is able to view highly magnified images 18 and 118, e.g., of a retina 25, via the display screen 20 after proper positioning of the optical head 170. A smaller additional display screen 200 could be positioned elsewhere in the ophthalmic surgical suite 10 to facilitate viewing by other medical personnel when assisting the surgeon.

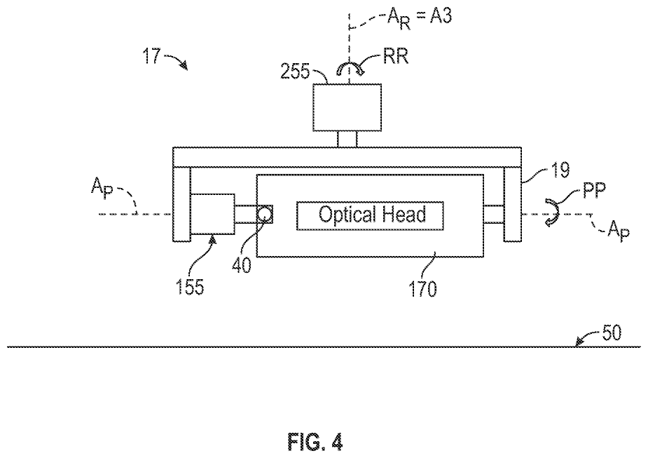

The optical head 170 in this example thus acts as a payload when the optical head 170 is securely connected to the distal end E1 of the SCARA 15, e.g., via a camera bracket 19. Gravity (arrow GG) acts on the SCARA 15 and the connected optical head 170 when the digital microscope 17 is positioned above the operating table 14 as shown. As noted below, a feature of the present solution is that none of the various joints J1-, J2, and J3 described below act in an anti-gravity mode. That is, each respective axis of rotation of the three revolute joints of the SCARA 15 is perpendicular to the floor 50, i.e., a horizontal plane. The optical head 170 of the microscope 17 as described below with reference to FIG. 4 has a pitch axis A_{P} that is arranged parallel to a plane of the floor 50. The pitch joint axis for the optical head 170 is located at the center of mass of the optical head 170, and therefore there is no rotational force due to gravity (arrow GG). The four-bar mechanism 400 described herein uses a linear actuator that is not back drivable to move the optical head 170 up/down against the gravitational force. If the three axes of the SCARA 15 were configured as passive axes, a gas spring could be used for gravity compensation.

As appreciated in the art, an ophthalmic microscope such as the microscope 17 illustrated in FIG. 1 consists of several main components. The optical head 170 contains the various lenses and optics for magnifying and illuminating a patient's eye during a given procedure. Although omitted from FIG. 1 for illustrative simplicity, the optical head 170 may contain an objective lens and zoom section providing different magnifications, a pair of eyepieces through which the surgeon views magnified images of the eye, and a controllable light source. The collective weight of such lenses and other hardware of the optical head 170 must be supported by the SCARA 15, even during a loss of power to the robotic system 11, while achieving the desired motion and positioning capabilities contemplated herein.

Also present within the exemplary ophthalmic surgical suite 10 of FIG. 1 is a cabinet 22 and an electronic control unit (ECU) 23. The ECU 23, e.g., one or more computer devices equipped with a computer-readable storage medium, processors, and other application-suitable hardware and software, is typically configured for coordinating electronic features and settings of the optical head 170 and/or other equipment or payloads used within the suite 10, e.g., foot pedals, hand controls, filters, video cameras, beam splitters, etc. Control of the robotic system 11 via the ECU 23 occurs by transmitting electronic control signals (CC₁₁) to one or more actuators as described below. Such a cabinet 22 could be constructed of a lightweight and easily sanitized construction, e.g., painted aluminum or stainless steel, and used to protect constituent hardware of the ECU 23 from possible ingress of dust, debris, and moisture.

Referring now to FIG. 2, the SCARA 15 is depicted in top view, and includes a plurality of revolute joints-respective first, second, and third revolute joints 30A (J1), 30B (J2), and 30C (J3). The first, second, and third revolute joints 30A, 30B, and 30C, which are collectively referred to as revolute joints 30 for simplicity, are included in the construction of the SCARA 15 along with additional fourth and fifth revolute joints 30D (J4) and 30E (J5) as depicted in FIG. 3. The SCARA 15 also includes respective first and second links 40A (L1) and 40B (L2) joined together by the second revolute joint 30B.

In a possible implementation, the bracket 19 of FIG. 1 is mounted to the distal end E1 of the second link 40B, with the optical head 170 securely mounted to the bracket 19. The respective first and second links 40A and 40B could be constructed of a powdered metallurgy alloy-like material constructed using cold isostatic pressing, and possibly containing aluminum and beryllium, such as but not limited to the commercially-available AlBeMet® AM162. Such materials are advantageous due to its high modulus to density ratio, which is almost 4× that of steel, aluminum, or titanium.

In the illustrated serial architecture of FIG. 2, the first link 40A of the SCARA 15 is connected to the base 13 via the first revolute joint 30A. The second link 40B is connected to the first link 40A via the second revolute joint 30B as noted above. Additionally, the second link 40B includes or terminates in the above-noted distal end E1 (see FIG. 1), which in turn is connected to the third revolute joint 30C as shown. Unlike the first link 40A, the second link 40B of FIG. 2 is constructed as a 1-DOF four-bar mechanism 400, an example implementation of which will now be described with reference to FIG. 3.

Referring briefly to FIG. 3, the four-bar mechanism 400 as contemplated herein for use as the second link 40B includes parallel horizontal links 40B-1 and 40B-2 coupled to parallel vertical links 40B-3 and 40B-4. On the orientation shown in FIG. 3, the horizontal links 40B-1 and 40B-2 are “horizontal” in the sense of being arranged parallel to the floor 50 and mutually parallel. Likewise, the two vertical links 40B-3 and 40B-4 are “vertical” in the sense of being arranged orthogonally to the floor 50, i.e., normal to the horizontal links 40B-1 and 40B, and likewise mutually parallel. The four constituent links 40A, 40B, 40C, and 40D of the four-bar mechanism 400 are joined together via four revolute joints 30, in this case the respective first and second revolute joints 30A and 30B along with two additional revolute joints, i.e., the fourth and fifth revolute 30D and 30E. The revolute joints 30A, 30B, 30C, and 30D of the illustrated four-bar mechanism 400, i.e., joints J1, J2, J3, and J4, are constructed such that the horizontal links 40B-1 and 40B-2 travel together in parallel planes, as appreciated in the art.

Up-down motion of the four-bar mechanism 400 is provided herein in one or more embodiments by a linear actuator 44, e.g., a non-back-drivable vertical harmonic drive unit. The linear actuator 44 may be coupled to the third revolute joints 30C and 30D FIG. 3, such that a translational force imparted by the linear actuator 44, as indicated by double-headed arrow LL, is used to help position the four-bar mechanism 400 and any connected load, e.g., the microscope 17 of FIG. 1. Advantages of using the linear actuator 44 include minimal backlash and back-drivability, the latter being of particular benefit in a power failure condition during which the structure of the de-energized SCARA 15 must support the full weight of the microscope 17. The linear actuator 44 is configured to control vertical motion of the microscope 17 when power is available, with such vertical motion relative to the surgeon's normal frame of reference being indicated by double-headed arrow VV. In the contemplated construction, none of the revolute joints 30A, 30B, 30C, and 30D of the four-bar mechanism 400 functions in an anti-gravity mode.

Referring once again to FIG. 2, an optional gas spring counterbalance device 33 may be operatively connected to the second link 40B to compensate for gravitational force. Air or other gas spring counterbalance devices, also referred to in the art as pneumatic counterbalance systems, are mechanical devices that employ a compressed gas to offset the weight of a load, in this case the optical head 170 of the microscope 17 shown in FIG. 1. To this end, the gas spring counterbalance device 33 provides a counterbalancing force to help hold optical head 170 in a static position. In a possible construction, the gas spring counterbalance device 33 may include a pneumatic cylinder or gas spring 37 connected to the second link 40B, and a source of compressed gas 39. The gas spring counterbalance device 33 can therefore be used to support the position-controlled SCARA 15 by providing a smooth and consistent counterbalancing force at a level sufficient for offsetting the collective weight of the SCARA 15 and optical head 170. Such a counterbalance force would be available through the full range of motion of the SCARA 15.

In accordance with an aspect of the disclosure, the first three revolute joints 30A, 30B, and 30C, i.e., joints J1, J2, and J3, respectively, may be individually powered by corresponding harmonic drive units 55A, 55B, and 55C (respectively labeled D1, D2, and D3). Each revolute joint 30A, 30B, and 30C rotates about a corresponding joint axis A1, A2, or A3. That is, the harmonic drive units 55A, 55B, and 55C are configured to power a respective one of the revolute joints 30A, 30B, and 30C in an optional actuated/actively-driven as opposed to passive construction of the SCARA 15. For instance, the revolute joints 30A, 30B, and 30C may be driven by one or more harmonic drive rotary actuators for high force and improved positional accuracy. In certain embodiments, one or more of the harmonic drive actuators may be configured as a slotless brushless DC (BLDC) rotary motor, which in this or other embodiments may include neodymium iron boron (NdFeB) magnets or other application suitable rare-earth magnets or non-rare-earth alternatives. A harmonic drive unit as used herein may include a compact precision mechanical speed reducer providing a high gear reduction ratio, e.g., at least about 50:1 to 100:1 or more in possible implementations of the SCARA 15.

Using flexible toothed components and an integrated motor and bearing configuration, a typical harmonic drive unit is able to achieve smooth and precise motion control. As such control occurs with minimal backlash, the harmonic drive units 55A, 55B, and 55C illustrated in FIG. 2 are particularly well-suited to the present surgical applications. However, those of ordinary skill in the art will appreciate that other actuators providing similar performance advantages may be used in alternative constructions of the SCARA 15, and therefore the SCARA 15 of FIG. 2 is not limited to use with the harmonic drive units 55A, 55B, and 55C for its actuation. For instance, one may power the revolute joints 30A, 30B, and/or 30C using, e.g., planetary, cycloidal, worm, or spur gear boxes, belt drive systems, rack-and-pinion devices, etc., without departing from the intended scope of the present disclosure.

Referring now to FIG. 4, the optical head 170 of the microscope 17 shown in FIG. 1 may be connected to or integrally include a sensor 40, e.g., an incremental or absolute encoder for position sensing. Measurements from such a sensor 40 or multiple such sensors 40 could be used by the ECU 23 of FIG. 1 to control operation of any of the harmonic drive units 55 of FIG. 2 and/or the linear actuator 44 of FIG. 3. This occurs using the electronic control signals (CC₁₁) depicted in FIG. 1. The optical head 170 is freely moveable about its pitch axis A_{P} via another harmonic drive unit 155, with the pitch axis A_{P} being arranged parallel to a plane of the floor 50 as noted above, with the base 13 of FIG. 1 resting on the floor 50 and possibly fixedly mounted thereto. Rotation of the optical head 170 may be actively driven about a rotation axis A_{R} by another harmonic drive unit 255, which for its part is arranged on the axis A3 of the revolute joint 30C shown in FIG. 2.

Pitch (PP) and rotation (RR) of the optical head 170 are selectively lockable, e.g., using stops, brakes, and/or motorized harmonic drives (not shown). The pitch and rotation of the optical head 170 are adjusted as needed to achieve desired imaging angles or orientations, with pitch as used herein referring to angular motion of the optical head 170 around the pitch axis A_{P}. In contrast, rotation as used herein refers to angular motion of the optical head 170 about its optical axis (not shown), with this axis coinciding with the rotational moment of the optical head 170. Those skilled in the art will appreciate that mechanical adjustments may be made by the surgeon as needed so as to set the desired pitch and rotation angles.

The robotic system 11 of FIG. 1 with its SCARA 15, i.e., the SCARA 15 and a 4-bar mechanism 400 together providing 4-DOF, thus enables an improved 4-DOF serial implementation with kinematics specific to cataract and VR surgical applications. The SCARA 15 as envisioned herein enables large amplitude, precise horizontal (xy) motion parallel to the floor 50. Using the four-bar mechanism 400 shown in FIG. 3, vertical (z) motion is permitted while preserving vertical pose of all three axes of the SCARA 15 that define its horizontal and vertical motion. Among the many attendant benefits of the present teachings, the SCARA 15 when configured as set forth above avoids interference between the SCARA 15 and the surgeon or patient when the display screen 20 of FIG. 1 is positioned directly in front of the surgeon.

Non-back drivable construction precludes drop of the SCARA 15 in the event of a power failure, with the presented itch axis AP of the optical head 170 as shown in FIG. 4 being ideal for visualization during, e.g., microinvasive glaucoma surgery (MIGS) surgery, as well as when inspecting and treating the retinal periphery. These and other attendant benefits will be readily appreciated by those skilled in the art in view of the foregoing disclosure.

As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Certain terminology may be used in the following description for the purpose of reference only, and thus are not intended to be limiting. For example, terms such as “above” and “below” refer to directions in the drawings to which reference is made. Terms such as “front,” “back,” “fore,” “aft,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

The detailed description and the drawings are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims.

Furthermore, the embodiments shown in the drawings or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

What is claimed is:

1. A robotic system for use with a microscope, comprising:
a base;
a support column; and
a selective-compliance-articulated robot arm (SCARA) connected to the base via the support column, including:
a plurality of revolute joints, including a first revolute joint, a second revolute joint, and a third revolute joint;
a first link connected to the base via the first revolute joint;
a second link connected to the first link via the second revolute joint, having a distal end connected to the third revolute joint, and configured as a four-bar mechanism, wherein the second link is configured to connect to the microscope; and
a linear actuator connected to the second link and configured to control vertical motion thereof, wherein none of the revolute joints of the SCARA functions in an anti-gravity mode.

2. The robotic system of claim 1, further comprising:
an electronic control unit (ECU) configured to provide electronic control signals to the linear actuator to control the vertical motion thereof.

3. The robotic system of claim 1, further comprising:
a plurality of harmonic drive units, wherein at least one of the revolute joints of the SCARA is configured to be powered by a corresponding one of the harmonic drive units.

4. The robotic system of claim 3, wherein at least one of the harmonic drive units is configured as a brushless DC motor.

5. The robotic system of claim 1, further comprising:
the microscope; and
a bracket mounted to the distal end of the second link, wherein the microscope is mounted to the bracket.

6. The robotic system of claim 5, wherein the base is positioned on a floor, and wherein the microscope has an optical head having a pitch axis arranged parallel to the floor.

7. The robotic system of claim 6, wherein pitch and rotation of the optical head are selectively lockable.

8. The robotic system of claim 6, further comprising:
a position sensor connected to the optical head and in communication with an electronic control unit (ECU).

9. The robotic system of claim 1, wherein the linear actuator includes a non-back drivable vertical harmonic drive unit.

10. The robotic system of claim 1, further comprising:
a gas spring counterbalance device operatively connected to the second link.

11. The robotic system of claim 1, wherein the first link and the second link are constructed of aluminum and beryllium.

12. A selective-compliance-articulated robot arm (SCARA) for use with a microscope and a base, comprising:
a plurality of revolute joints, including a first revolute joint, a second revolute joint, and a third revolute joint;
a first link connectable to the base via the first revolute joint;
a second link connected to the first link via the second revolute joint, having a distal end connected to the third revolute joint, and configured as a four-bar mechanism, wherein the second link is configured to connect to the microscope; and
a linear actuator connected to the second link and configured to control vertical motion thereof in response to electronic control signals from an electronic control unit (ECU), wherein the linear actuator includes a non-back drivable vertical harmonic drive unit, and wherein none of the revolute joints of the SCARA functions in an anti-gravity mode.

13. The SCARA of claim 12, further comprising:
a harmonic drive unit, wherein one of the revolute joints of the SCARA is powered by the harmonic drive unit.

14. The SCARA of claim 13, wherein the harmonic drive unit includes a plurality of harmonic drive units, and wherein each of the revolute joints of the SCARA is individually driven by a corresponding one of the harmonic drive units.

15. The SCARA of claim 12, further comprising:
a bracket mounted to the distal end of the second link, wherein a microscope is mounted to the bracket.

16. The SCARA of claim 15, wherein the microscope is a digital or analog ophthalmic microscope having an optical head.

17. The SCARA of claim 16, wherein the base is configured to be positioned on a floor, and wherein the optical head has a pitch axis arranged parallel to the floor.

18. The SCARA of claim 16, further comprising:
a position sensor connected to the optical head and in communication with the ECU.

19. The SCARA of claim 12, further comprising:
a gas spring counterbalance device operatively connected to the second link.

20. A robotic system, comprising:
an ophthalmic microscope;
a bracket mounted to the microscope;
a base;
a support column;
an electronic control unit (ECU) configured to output electronic control signals; and
a selective-compliance-articulated robot arm (SCARA) connected to the base via the support column, including:
a plurality of revolute joints powered by one or more harmonic drive units, the revolute joints including a first revolute joint, a second revolute joint, and a third revolute joint;
a first link connected to the base via the first revolute joint;
a second link connected to the first link via the second revolute joint, having a distal end connected to the third revolute joint, and configured as a four-bar mechanism, wherein the second link is connectable to the microscope via the bracket;
a gas spring counterbalance device operatively connected to the second link; and
a linear actuator connected to the second link and configured to control vertical motion thereof in response to the electronic control signals, wherein none of the revolute joints of the SCARA functions in an anti-gravity mode.
